(19) United States
(12) Patent Application Publication
(10) Pub. No.: US 2005/0214884 A1
(43) Pub. Date: Sep. 29, 2005

(54) Method for stabilizing cholesterol dehydrogenase, cholesterol dehydrogenase-containing composition and cholesterol measuring reagent

(75) Inventors: Yasuhiro Sakai, Kobe-shi (JP); Kenji Isshiki, Kobe-shi (JP); Kouji Kishi, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION

(21) Appl. No.: 10/995,383

(22) Filed: Nov. 24, 2004

(30) Foreign Application Priority Data
Nov. 28, 2003 (JP) 2003-398455
Sep. 29, 2004 (JP) 2003-284317

(51) Int. Cl. C12Q 1/60; C12N 9/02

(52) U.S. Cl. 435/11; 435/189

(57) ABSTRACT

The present invention is to provide a novel method for stabilizing cholesterol dehydrogenase. Also, the present invention is to provide a novel cholesterol-containing composition and a cholesterol measuring reagent.

### FIELD OF THE INVENTION

The present invention relates to a method for stabilizing cholesterol dehydrogenase, a cholesterol dehydrogenase-containing composition and a cholesterol measuring reagent.

### BACKGROUND

Previously, many methods for measurement using an enzyme have been developed due to excellent specificity of a reaction and reproductivity, and simple procedure. In particular, in the field of a clinical test, many methods are known in measuring components in a blood sample. Meanwhile, in the field of a clinical test, recently, a lipid test has been increased and, in particular, cholesterol is important as a risk factor for arteriosclerosis, which is an adult disease, and a test of cholesterol has been increased. As a method for measuring cholesterol, a method using an enzyme is currently general and, as the method, a method for using cholesterol oxidase and a method for using cholesterol dehydrogenase are known (Japanese Patent Application Laid-open No.5-176797). In the former method, a step of reacting hydrogen peroxide produced by an enzymatic reaction with a color developing substrate in the presence of peroxidase to lead to a quinone pigment is necessary, and procedures are complicated. In addition, there is a defect that an error is caused by bilirubin or ascorbic acid. To the contrary, the latter method measures an amount of NAD(P) produced by an enzymatic reaction between cholesterol and cholesterol dehydrogenase and, since measurement can be performed only by measuring an absorbance of produced NAD(P)H, the method has an advantage that the method is simple, and there is little influence of debrises in a sample.

However, cholesterol dehydrogenase is an unstable enzyme, and various devices are tried in order to prepare a measurement reagent.

For example, there are provided a method for stabilizing a physiologically active protein using crystalline (Japanese Patent Application Laid-open No.7-236483), a method of stabilizing cholesterol dehydrogenase using a glycoside (Japanese Patent Application Laid-open No.9-313178), and a method of stabilizing a physiologically active substance using a chelating agent (Japanese Patent Application Laid-open No.2001-299385).

### SUMMARY

An object of the present invention is to provide a novel method for stabilizing cholesterol dehydrogenase, which is different from the previous method for stabilizing cholesterol dehydrogenase. Also, an object of the present invention is to provide a novel cholesterol-containing composition and a cholesterol measuring reagent.

A first aspect of the present invention relates to a method for stabilizing a cholesterol dehydrogenase in a cholesterol dehydrogenase-containing composition, the method comprising; adding a glycine compound represented by the following formula (1) to the cholesterol dehydrogenase-containing composition;
R—(NH—CH₂—CO)ₙ—NH—CH₂—COOH (1)
wherein R represents hydrogen, an alkyl group which may have a substituent, a phenyl group which may have a substituent or a carbonyl group which may have a substituent and n represents 0 to 2.

Also, a second aspect of the present invention relates to a cholesterol dehydrogenase-containing composition, which contains a glycine compound represented by the above chemical formula (1) and cholesterol dehydrogenase.

Further, a third aspect of the present invention relates to a cholesterol measuring reagent, which comprises a glycine compound represented by the above chemical formula (1), cholesterol dehydorgenase and a coenzyme.

### DESCRIPTION OF THE PREFERRED EMBODIMENT

A cholesterol dehydrogenase-containing composition comprises cholesterol dehydrogenase and a glycine compound represented by the chemical formula (1).
R—(NH—CH₂—CO)ₙ—NH—CH₂—COOH (1)
(wherein R represents hydrogen, an alkyl group optionally having a substituent, a phenyl group optionally having a substituent or a carbonyl group optionally having a substituent and n represents 0 to 2).

Cholesterol dehydrogenase is not particularly limited, but examples include cholesterol dehydrogenases derived from microorganisms, animals and plants, preferably cholesterol dehydrogenases derived from microorganisms. Cholesterol dehydrogenase derived from Nocardia sp. among microorganisms is preferable. In addition, cholesterol dehydrogenase obtained by culturing these microorganisms, and purifying this, and cholesterol dehydrogenase obtained by recombination may be used.

A glycine compound used for stabilizing cholesterol dehydrogenase is represented by the following chemical formula (1):
R—(NH—CH₂—CO)ₙ—NH—CH₂—COOH (1)
(wherein R represents hydrogen, an alkyl group optionally having a substituent, a phenyl group optionally having a substituent or a carbonyl group optionally having a substituent and n represents 0 to 2).

Herein, R is an alkyl group optionally having a substituent, a phenyl group optionally having a substituent, or a carbonyl group optionally having a substituent. Examples of an alkyl group include a methyl group and an ethyl group. Examples of a phenyl group include a hydroxyphenyl group. Examples of the substituent include a hydroxymethyl group, a hydroxyl group, an amino group, a carboxyl group, a nitro group, a methoxy group, and a thiol group. Two or more kinds of glycine compounds represented by the chemical formula (1) may be combined. In addition, preferable examples of the glycine compound represented by the chemical formula (1) include glycine, glycylglycine and tricine. These glycine compounds have buffering action, and they may exert a role as a buffering agent.

Other stabilizing substance such as cholic acid, glycoside, adenosine monophosphate, crystallin and chelating agent and derivatives thereof together with the glycine compound represented by the above chemical formula (1) may be added to a cholesterol dehydrogenase-containing composition. As cholic acid or a derivative thereof, there can be exemplified salts of cholic acid (e.g. sodium salt), deoxycholic acid or salts thereof (e.g. sodium salt), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), and N,N-bis(3-D-gluconeamidopropyl)cholamide (deoxy-BIGCHAP). As a glycoside or a derivative thereof, there can be exemplified n-dodecyl-β-D-maltoside (dodecylmaltose), n-heptyl-β-D-thioglucoside, n-octyl-β-D-glucoside, n-octyl-β-D-thioglucoside, digitonin, sucrose monocaprate, sucrose monolaurate, 2-ethyl-hexylglucoside, n-octanoyl-N-methylglucamide, n-methylglucamide, n-nonanoyl-N-methylglucamide and n-decanoyl-N-methylglucamide. Further, as adenosine monophosphate or a derivative thereof, there can be exemplified adenosine monophosphate and salts thereof (sodium salt, potassium salt etc.). In addition, as crystallin or a derivative thereof, there can be exemplified α-crystallin, β-crystallin, γ-crystallin, and δ-crystallin. In addition, as a chelating agent, there can be exemplified ethylenediaminediacetate (EDDA), iminodiacetate (IDA), nitrilotriacetic acid (NTA), hydroxyethyliminodiacetic acid (HIDA), ethylenediaminedipropionic acid (EDDP), ethylenediaminetetrakismethylenephosphoric acid (EDTPO), hydroxylethylethylenediaminetetraacetic acid (EDTA-OH), diaminopropanoltetraacetic acid (DPTA-OH), nitrilotrismethylenephosphoric acid (NTPO), bis(aminophenyl)ethyleneglycoltetraacetic acid (BAPTA), nitrilotripropionic acid (NTP), dihydroxyethylglycine (DHEG), and glycoletherdiaminetetraacetic acid (GEDTA).

Effect of stabilizing cholesterol dehydrogenase due to the glycine compound represented by the chemical formula (1) is such that the cholesterol dehydrogenase-containing composition exerts remarkable effect in the liquid state, and also exert remarkable effect in the lyophilized state. An amount of the glycine compound represented by the chemical formula (1) to be added to the cholesterol dehydrogenase-containing composition can be appropriately set depending on a kind thereof, a content of cholesterol dehydrogenase in the cholesterol dehydrogenase-containing composition, and condition of storing the cholesterol dehydrogenase-containing composition. For example, a concentration of the glycine compound to be added to the cholesterol dehydrogenase-containing composition is preferably 0.001 to 200 mM, more preferably 10 to 1500 mM, further preferably 100 to 1000 mM.

The cholesterol dehydrogenase-containing composition can be used for measuring lipoprotein cholesterol such as high-density lipoprotein (HDL) cholesterol, low-density lipoprotein (LDL) cholesterol or very low-density lipoprotein (VDLD) cholesterol, in addition to measuring total cholesterol or free cholesterol.

A total cholesterol measuring reagent comprises a first reagent comprising a coenzyme necessary for a reaction of a cholesterol dehydrogenase (hereinafter, abbreviated as coenzyme), cholesterol releasing enzyme and a reaction promoter, and a second reagent comprising a cholesterol dehydrogenase-containing composition. In addition, a free cholesterol measuring reagent comprises a first reagent comprising a coenzyme and a reaction promoter, and a second reagent comprising a cholesterol dehydrogenase-containing composition. Examples of the coenzyme include β-nicotineamide adenine dinucleotide oxidized-type (NAD), thio-nicotineamide adenine dinucleotide oxidized-type (t-NAD), β-nicotineamide adenine dinucleotide phosphate oxidized-type (NADP), and thio-nicotineamide adenine dinucleotide phosphate oxidized-type (t-NADP). By the presence of cholesterol, these coenzymes are converted into respective reduced-types, that is, NADH, t-NADH, NADPH or t-NADPH.

The reaction promoter is not particularly limited as far as it can block a ketone group of Δ4-cholestenone which is a substance obtained by oxidation of cholesterol with cholesterol dehydrogenase, and examples include hydrazine, a hydrate thereof, a salt thereof, and a derivative thereof. The hydrazine derivative corresponds to a compound having a basal skeleton of hydrazyl. As a specific example of the hydrazine hydrate, hydrazine (monohydrate) is preferable and, as the hydrazine derivative, hydrazinium dichloride, hydrazinium nonobromide, hydrazinium sulfate are suitable. Other examples include phenylhydrazinium chloride, phenylhydrazine-p-sulfonic acid, phenylhydrazinium sulfate, and hydradinepyridine.

An amount of hydrazine, a hydrate thereof a salt thereof, or a derivative thereof to be added is different depending on a kind thereof, a composition thereof, and other conditions, and is usually 5 to 500 mM, preferably 20 to 200 mM as a concentration when a sample, a first reagent and a second reagent are mixed.

The cholesterol releasing enzyme is not particularly limited as far as it has activity of releasing cholesterol which is ether-bound with a lipoprotein, and examples include cholesterol esterase, and lipoprotein lipase.

The lipoprotein cholesterol measuring reagent comprises a first reagent comprising a reaction controlling substance and a coenzyme, and a second reagent comprising a cholesterol dehydrogenase-containing composition and a cholesterol releasing enzyme.

As the lipoprotein, there are HDL, LDL, VLDL, CM and remnant-like lipoprotein. By adding a reaction controlling substance to form a complex with these lipoproteins, these lipoprotein cholesterols can be measured.

Examples of the reaction controlling substance include calixarene, polyethylene glycol (PEG), phosphotungstic acid, dextran sulfate, and heparin. Further, these substances may be used by combining with a cation such as Mg⁺⁺, Mn⁺⁺, Ca⁺⁺, Li⁺⁺ and Ni⁺⁺.

As the reaction controlling substance, calixarene is preferable. A method for measuring a lipoprotein cholesterol using calixarene will be explained below.

Calixarene is a cyclic oligomer in which 4 to 8 molecules of phenol is polymerized in a ring manner with a methylene group. Examples of calixarene include calix(4)arene, calix(6)arene, calix(8)arene, calix(4)arene sulfate, calix(6)arene sulfate, calix(8)arene sulfate, calix(4)arene acetate, calix(6)arene acetate, calix(8)arene acetate, carboxycalix(4)arene, carboxycalix(6)arene, carboxycalix(8)arene, calix(4)areneamine, calix(6)areneamine, and calix(8)areneamine. One kind or two or more kinds selected from these calixarenes can be used.

As the coenzyme or the cholesterol releasing enzyme, the aforementioned coenzymes or cholesterol releasing enzyme can be used.

A concentration of calixarene in a first reagent can be experimentally determined by respective measuring conditions since its optimal concentration is changed depending on a kind of a lipoprotein, a kind of a calixarene and measuring condition such as pH.

Optionally, in order to scavenge free cholesterol or a cholesterol in lipoprotein which is not to be measured, cholesterol oxidase or cholesterol dehydrogenase may be added to a first reagent. Preferably, cholesterol oxidase is used.

For example, when cholesterol in HDL is measured, calixarene is added in a first reaction to form a complex of a lipoprotein other than HDL with calixarene, to stabilize it, and an enzyme is added in a second reaction, and a concentration of cholesterol in HDL may be measured.

When cholesterol in LDL is measured, the aforementioned conditions are combined, and a complex of LDL and calixarene is formed in a first reaction to stabilize it, while free cholesterol and cholesterol in HDL and VLDL are pre-reacted to scavenge them, and cholesterol in remaining LDL is measured in a second reaction.

When cholesterol in VLDL is measured, the aforementioned conditions are combined, and a complex of VLDL and calixarene is formed in a first reaction to stabilize it, while cholesterol in HDL and LDL is pre-reacted to scavenge it, and cholesterol in remaining VLDL is measured in a second reaction.

Examples of a sample include serum, plasma, urine, saliva and semen.

### EXAMPLE

Examples are presented below, but the present invention is not limited to them.

### Example 1

A loading solution (pH 8.8) containing 0.7 U/mL cholesterol dehydrogenase, 0.5M compound shown in Table 1, and 2 mg/mL sodium cholate was prepared, allowed to stand at 37° C. for 3 days, and remaining activity of cholesterol dehydrogenase was obtained by the method shown below. Results of an amount of remaining activity after storage expressed as a relative value letting enzyme activity immediately after sample preparation to be 100% are shown in Table 1. As shown in Table 1, it was made clear that, by adding a glycine compound such as glycine, glycylglycine and tricine, an enzyme is stabilized.

1. Method for Measuring Activity of Cholesterol Dehydrogenase

A reagent having the fallowing composition was prepared.

Reagent A:

| | |
|---|---|
| Cholesterol | 1 mg/mL |
| Triton X-100 | 20 mg/mL |

Reagent B:

| | |
|---|---|
| β-NAD | 3 mg/mL |
| Tris buffer (pH 8.5) | 0.3 M |

Diluent solution:

| | |
|---|---|
| Sodium cholate | 3 mg/mL |
| Phosphate buffer (pH 7.0) | 20 mM |

2. Measuring Method

A loading solution was diluted 10-fold with a diluent, which was used as a sample solution. 10 μL of a sample solution was mixed with 200 μL of the reagent A, and the mixture was maintained at 25° C. for 5 minutes. 100 μL of the reagent B was added to the maintained mixture, and an amount of change in an absorbance per minute at 25° C. and 340 nm was obtained. Similarly, instead of a sample solution, 10 μL of a diluent was added, and a reagent blank was obtained.

TABLE 1

| Reagent name | Remaining rate (%) |
|---|---|
| Glycine | 52 |
| Glycylglycine | 50 |
| Tricine | 48 |
| TAPS | 28 |
| Bicine | 23 |
| HEPES | 15 |
| CHES | 1 |
| Tris(hydroxymethyl)aminomethane | 4 |
| 2-Amino-2-methyl-1,3-propanediol | 0 |
| Diethanolamine | 1 |
| Potassium pyrophosphate | 9 |
| Boric acid | 9 |

### Example 2

Concentrations of compounds shown in Table 2 were studied. According to the same manner as that of Example 1 except that a concentration of each compound was changed in 0.1 to 0.5 M, study was performed. Results are shown in Table 2. As shown in Table 2, sufficient stabilizing effect was confirmed in a range of 0.1 to 0.5 M.

TABLE 2
Unit: remaining rate (%)

| | Storage at 37° C. for 2 days | | | | |
|---|---|---|---|---|---|
| Reagent name | 0.5M | 0.4M | 0.3M | 0.2M | 0.1M |
| Glycine | 66 | 59 | 53 | 48 | 31 |
| Glycylglycine | 51 | 47 | 41 | 39 | 38 |
| Tricine | 53 | 47 | 41 | 35 | 30 |
| TAPS | 35 | 32 | 29 | 28 | 27 |
| 2-Amino-2-methyl-1, | 1 | 0 | 2 | 3 | 9 |
| 3-propanediol | | | | | |
| Diethanolamine | 1 | 1 | 1 | 1 | 6 |
| Potassium pyrophosphate | 16 | 13 | 7 | 1 | 0 |

### Example 3

Stabilization of cholesterol dehydrogenase by combined use of glycylglycine and glycine was studied.

2 U/mL cholesterol dehydrogenase, 14 mM dodecylmaltose, 0.5 M glycylglycine and each concentration of glycine were added to prepare a loading solution (pH 8.8), and a remaining rate of cholesterol dehydrogenase after storage at 37° C. for a predetermined term was studied. Remaining activity of cholesterol dehydrogenase was measured by the method shown in Example 1.

Results of an amount of remaining activity after storage expressed as a relative value letting enzyme activity immediately after preparation of a loading solution to be 100% are shown in Table 3. As shown in Table 3, it was made clear that, by adding glycine, stabilization effect is further improved.

TABLE 3
Unit: remaining rate (%)

| Glycylglycine | Glycine | | |
|---|---|---|---|
| (M) | (M) | 37° C., 1 day | 37° C., 2 day |
| 0.5 | 0.00 | 66.7 | 54.0 |
| 0.5 | 0.05 | 67.8 | 73.8 |
| 0.5 | 0.10 | 69.3 | 62.2 |
| 0.5 | 0.20 | 72.1 | 67.0 |
| 0.5 | 0.30 | 74.3 | 65.8 |
| 0.5 | 0.40 | 75.8 | 68.5 |
| 0.5 | 0.50 | 77.3 | 71.0 |
| 0.5 | 0.60 | 79.4 | 80.2 |

### Example 4

Stabilization of cholesterol dehydrogenase by combined use of dodecylmaltose that is a glycoside, and adenosine monophosphate (AMP) was studied.

Each of various concentrations of dodecylmaltose and AMP were added to 2 U/mL cholesterol dehydrogenase, 0.5 M glycylglycine and 0.5 M glycine to prepare a loading solution (pH 8.8), and a remaining rate of cholesterol dehydrogenase after storage at 37° C. for a predetermined term was studied.

Results of an amount of remaining activity after storage expressed as a relative value letting enzyme activity immediately after preparation of a sample to be 100% are shown in Table 4. As shown in Table 4, it was made clear that, by adding dodecylmaltose and AMP, stabilization effect is further improved.

TABLE 4
Unit: remaining rate (%)

| | | | 37° C., | |
|---|---|---|---|---|
| Dodecylmaltose | AMP | 37° C., 1 day | 2 day | 37° C., 3 day |
| 5.9 mM | 5 mM | 84.2 | 75.7 | 69.9 |
| | 7.5 mM | 84.9 | 78.1 | 73.1 |
| | 10 mM | 87.5 | 80.5 | 76.4 |
| 7.8 mM | 5 mM | 88.8 | 82.3 | 77.8 |
| | 7.5 mM | 91.0 | 86.1 | 80.4 |
| | 10 mM | 90.7 | 87.1 | 81.9 |
| 9.8 mM | 5 mM | 91.9 | 87.6 | 82.2 |
| | 7.5 mM | 91.8 | 87.0 | 83.4 |
| | 10 mM | 93.9 | 90.3 | 85.2 |

### Example 5

Stabilization of Total Cholesterol Measuring Reagent

A total cholesterol measuring reagent using glycine as an agent for stabilizing cholesterol dehydrogenase is shown below. A first reagent of a total cholesterol measuring reagent is constructed of a solution (pH 7.0) containing 25 mM PIPES, 100 mM hydrazinium dichloride, 0.5% nonion A-10R, 0.5% Triton X-100, 2 mM sodium cholate, 5 mM β-NAD and 2 U/mL cholesterol esterase and a second reagent is constructed of a solution (pH 8.5) containing 200 mM glycine, 5 mM sodium cholate and 14 U/mL cholesterol dehydrogenase. As a control, a total cholesterol measuring reagent using TAPS instead of glycine in the aforementioned second reagent and not containing glycine was also prepared.

Stability of a total cholesterol measuring reagent using glycine was better than that of a total cholesterol measuring reagent not containing glycine.

### Example 6

Stability of HDL Cholesterol Measuring Reagent

A HDL cholesterol measuring reagent using glycine as an agent for stabilizing cholesterol dehydrogenase is shown below. A first reagent of the HDL cholesterol measuring reagent is constructed of a solution (pH 6.5) containing 50 mM HEPES, 80 mM hydrazinium dichloride, 2 mM calix(8)arene sulfate, 5.0 mM β-NAD and 0.5 U/mL of cholesterol oxidase and a second reagent is constructed of a solution (pH 8.5) containing 200 mM glycine, 20 U/mL cholesterol dehydrogenase and 6 U/mL of cholesterol esterase. As a control, a HDL cholesterol measuring reagent using TAPS instead of glycine in the aforementioned second reagent and not containing glycine was also prepared.

Stability of a HDL cholesterol measuring reagent using glycine was better than that of a HDL cholesterol measuring reagent not containing glycine.

### Example 7

Confirmation of Stability of LDL Cholesterol Measuring Reagent

A LDL cholesterol measuring reagent using glycine as an agent for stabilizing cholesterol dehydrogenase is shown below. A first reagent of the LDL cholesterol measuring reagent is constructed of a solution (pH 7.0) containing 50 mM HEPES, 1.2 mM calix(6)arene sulfate, 2 mM sodium cholate, 0.5 U/ml cholesterol oxidase, 5.0 mM β-NAD and 1 U/ml of cholesterol esterase and a second reagent is constructed of a solution (pH 8.5) containing 200 mM glycine, 300 mM hydrazinium dichloride and 20 U/ml cholesterol dehydrogenase. As a control, a LDL cholesterol measuring reagent using TAPS instead of glycine in the aforementioned second reagent and not containing glycine was also prepared.

Stability of a LDL cholesterol measuring reagent using glycine was better than that of a LDL cholesterol measuring reagent not containing glycine.

### Example 8

Stability of VLDL Cholesterol Measuring Reagent

A VLDL cholesterol measuring reagent using glycine as an agent for stabilizing cholesterol dehydrogenase is shown below. A first reagent of the VLDL cholesterol measuring reagent is constructed of a solution (pH 7.0) containing 50 mM HEPES, 10 mM calix(6)arene sulfate, 2 mM sodium cholate, 0.5 U/ml cholesterol oxidase, 5.0 mM β-NAD and 1 U/ml of cholesterol esterase and a second reagent is constructed of a solution (pH 8.5) containing 200 mM glycine, 300 mM hydrazinium dichloride and 20 U/ml cholesterol dehydrogenase. As a control, a VLDL cholesterol measuring reagent using HEPES instead of glycine of the aforementioned second reagent and not containing glycine was also prepared.

Stability of a VLDL cholesterol measuring reagent using glycine was better than that of a VLDL cholesterol measuring reagent not containing glycine.

1. A method for stabilizing a cholesterol dehydrogenase in a cholesterol dehydrogenase-containing composition, the method comprising;
adding a glycine compound represented by the following formula (1) to the cholesterol dehydrogenase-containing composition;
R—(NH—CH₂—CO)ₙ—NH—CH₂—COOH (1)
wherein R represents hydrogen, an alkyl group which may have a substituent, a phenyl group which may have a substituent or a carbonyl group which may have a substituent and n represents 0 to 2.

2. The method for stabilizing cholesterol dehydrogenase according to claim 1, wherein the glycine compound is selected from the group consisting of glycine, glycylglycine and tricine.

3. The method for stabilizing cholesterol dehydrogenase according to claim 1, comprising adding a component selected from the group consisting of glycoside, cholic acid and adenosine monophosphate, to the composition.

4. The method for stabilizing cholesterol dehydrogenase according to claim 1, wherein a concentration of the glycine compounds is 10 to 2000 mM.

5. A cholesterol dehydrogenase-containing composition, comprising a cholesterol dehydrogenase and a glycine compound represented by the following chemical formula (1);
R—(NH—CH₂—CO)ₙ—NH—CH₂—COOH (1)
wherein R represents hydrogen, an alkyl group which may have a substituent, a phenyl group which may have a substituent or a carbonyl group which may have a substituent and n represents 0 to 2.

6. The cholesterol dehydrogenase-containing composition according to claim 5, wherein the glycine compound is selected from the group consisting of glycine, glycylglycine and triglycine.

7. The cholesterol dehydrogenase-containing composition according to claim 5, comprising a compound selected from the group consisting of glycoside, cholic acid and adenosine monophosphate.

8. A cholesterol measuring reagent, comprising a cholesterol dehydrogenase, a coenzyme and a glycine compound represented by the following chemical formula (1),
R—(NH—CH₂—CO)ₙ—NH—CH₂—COOH (1)
wherein R represents hydrogen, an alkyl group which may have a substituent, a phenyl group which may have a substituent or a carbonyl group which may have a substituent and n represents 0 to 2.

9. The cholesterol measuring reagent according to claim 8, wherein the reagent is a total cholesterol measuring reagent comprising a first reagent and a second reagent, the first reagent comprises a coenzyme, cholesterol releasing enzyme and a reaction promoter and the second reagent comprises the glycine compound and the cholesterol dehydrogenase.

10. The cholesterol measuring reagent according to claim 8, wherein the reagent is a free cholesterol measuring reagent comprising a first reagent and a second reagent, the first reagent comprises a coenzyme and a reaction promoter and the second reagent comprises the glycine compound and the cholesterol dehydrogenase.

11. The cholesterol measuring reagent according to claim 8, wherein the reagent is a lipoprotein cholesterol measuring reagent comprising a first reagent and a second reagent, the first reagent comprises a reaction controlling substance and a coenzyme and the second reagent comprises the glycine compound, the cholesterol dehydrogenase and a cholesterol releasing enzyme.

12. The cholesterol measuring reagent according to claim 11, wherein the reaction controlling substance is at least one selected from the group consisting of calixarene, polyethylene glycol, phosphotungstic acid, dextran sulfate and heparin.

13. The cholesterol measuring reagent according to claim 12, wherein the reaction controlling substance is calixarene.

14. The cholesterol measuring reagent according to claim 11, wherein a concentration of the glycine compound contained in the second reagent is 10 to 2000 mM.

15. The cholesterol measuring reagent according to claim 11, wherein the lipoprotein cholesterol measuring reagent is a high-density lipoprotein (HDL) cholesterol measuring reagent, a low-density lipoprotein (LDL) cholesterol measuring reagent or a very low-density lipoprotein (VDLD) cholesterol measuring reagent.

16. A method for measuring a cholesterol in high-density lipoprotein, comprising the steps of;
mixing a sample and a first reagent comprising a reaction controlling substance and a coenzyme to form a complex of the reaction controlling substance and a lipoprotein other than a high-density lipoprotein,
mixing the resulting mixture and a second reagent comprising a cholesterol dehydrogenase, a cholesterol releasing enzyme and a glycine compound represented by the following formula (1);
R—(NH—CH₂—CO)ₙ—NH—CH₂—COOH (1)
wherein R represents hydrogen, an alkyl group which may have a substituent, a phenyl group which may have a substituent or a carbonyl group which may have a substituent, and n represents 0 to 2; and
measuring a cholesterol in high-density lipoprotein cholesterol of the second resulting mixture.

17. A method for measuring a cholesterol in low-density lipoprotein, comprising the steps of;
mixing a sample and a first reagent comprising a reaction controlling substance and a coenzyme to form a complex of the reaction controlling substance and a low-density lipoprotein,
scavenging cholesterol containing a lipoprotein other than the low-density lipoprotein in the resulting mixture,
mixing the scavenged mixture and a second reagent comprising a cholesterol dehydrogenase, a cholesterol releasing enzyme and a glycine compound represented by the following formula (1);
R—(NH—CH₂—CO)ₙ—NH—CH₂—COOH (1)
wherein R represents hydrogen, an alkyl group which may have a substituent, a phenyl group which may have a substituent or a carbonyl group which may have a substituent and n represents 0 to 2; and
measuring a cholesterol in low-density lipoprotein of the second resulting mixture.

18. A method for measuring a cholesterol in very low-density lipoprotein, comprising the steps of;
mixing a sample and a first reagent comprising a reaction controlling substance and a coenzyme to form a complex of the reaction controlling substance and a very low-density lipoprotein,
scavenging cholesterol containing a lipoprotein other than the very low-density lipoprotein in the resulting mixture,
mixing the scavenged mixture and a second reagent comprising a cholesterol dehydrogenase, a cholesterol releasing enzyme and a glycine compound represented by the following formula (1);
R—(NH—CH₂—CO)ₙ—NH—CH₂—COOH (1)
wherein R represents hydrogen, an alkyl group which may have a substituent, a phenyl group which may have a substituent or a carbonyl group which may have a substituent and n represents 0 to 2; and
measuring a cholesterol in very low-density lipoprotein of the second resulting mixture.
